(19) United States
(12) Patent Application Publication
(10) Pub. No.: US 2014/0256608 A1
(43) Pub. Date: Sep. 11, 2014

(54) FOAMING OIL

(71) Applicant: R.I.T.A. CORPORATION, Crystal Lake, IL (US)

(72) Inventors: Thomas Keech, Roselle, IL (US); April Ensinger, Huntley, IL (US); Katherine S. Meka, Inverness, IL (US); Daniel V. Beio, Huntley, IL (US)

(73) Assignee: R.I.T.A. CORPORATION, Crystal Lake, IL (US)

(21) Appl. No.: 14/178,842

(22) Filed: Feb. 12, 2014

Related U.S. Application Data
(60) Provisional application No. 61/773,335, filed on Mar. 6, 2013.

(51) Int. Cl. C11D 17/08; C11D 3/20; A61Q 1/14; A61K 8/04

(52) U.S. Cl. CPC C11D 17/08; A61K 8/046; C11D 3/2093; A61Q 1/14
USPC 510/136; 510/490; 510/488; 510/245; 510/109; 510/275

(57) ABSTRACT

Compositions comprising a high amount of a liquid oil, a lactylate, and at least one of an alkanolamide and a sarcosinate are disclosed. The high oil content effectively removes oily residues from a surface treated with the composition. The compositions are essentially free of water, but are water rinsable to effect easy and rapid removal of the composition from a surface.

### CROSS-REFERENCE TO RELATED APPLICATIONS

This application claims the benefit of U.S. provisional patent Application No. 61/773,335, filed Mar. 6, 2013, incorporated herein by reference in its entirety.

### FIELD OF THE INVENTION

The present invention relates to an essentially water-free composition that can be applied to a surface, then diluted on or rinsed from the surface with water. The compositions are (a) essentially free, or free, of water and harsh anionic surfactants that can irritate the skin, (b) contain a high percentage of a liquid oil for effective removal of oily residues on a surface, and (c) generate a substantial foam during rinsing for effective removal of the oil and residues from the surface and for consumer esthetics.

### BACKGROUND OF THE INVENTION

Cleaners for animate and inanimate surfaces typically are water-based compositions containing high-foaming anionic surfactants. The anionic surfactants act to emulsify and solubilize oily residues on treated surfaces, which allows the oily residues to be rinsed from the surface with the anionic surfactants. Such compositions have been used for decades, but the anionic surfactants are harsh to the skin, which leads to skin irritation.

For example, present day facial cleaners are designed to remove facial makeup and oily residues from the skin. However, they contain very harsh surfactants that can cut through these oily residues in one step, leaving the skin totally stripped of lipids resulting in a compromised skin barrier and exacerbated dry skin. Another conventional removal method is to use an oil-based makeup remover to dislodge skin debris by applying the makeup remover to create a continuous film, then dry wiping the skin with a paper tissue, and following with a water/surfactant based cleaner to remove the residual makeup remover and debris. Neither method is optimal.

The present invention is directed to overcoming problems associated with prior aqueous anionic surfactant-based cleaners, and especially skin cleaners and related skin product treatments.

### SUMMARY OF THE INVENTION

The present invention is directed to an essentially anhydrous composition that is phase stable during storage, is capable of removing oily residues and debris from a surface, and that is readily removed from the surface by a water rinsing. The compositions effectively remove oily residues and debris from a surface, such as the skin, due to its highly oleophilic nature. In addition, the compositions are easily removed from the treated surface with a simple water rinsing, which also generates a consumer acceptable foam.

Therefore, one aspect of the present invention is to provide a composition comprising a liquid oil, a lactylate, and at least one of an alkanolamide and a sarcosinate. The composition is essentially free of water, and in most preferred embodiments, the composition is free of water, i.e., is anhydrous.

A foaming oil composition of the present invention can be used “as is” or diluted with water. In accordance with the present invention, the cleaning properties are enhanced when the composition is used “as is”, or at a low dilution, such as diluted with an equal or less volume amount of water, i.e., a 50% or less volume dilution with water. For example, when used as a shaving composition, dilution provides a consumer-acceptable foam level for shaving and the composition is mild to the skin due to its high oil content.

Another aspect of the present invention is to provide a method of cleaning animate and inanimate surfaces comprising spraying or applying, e.g., rubbing, a composition of the present invention onto the surface, then rinsing the composition from the surface with water. In various embodiments, a present foaming oil composition is formulated as a facial cleaner, a facial scrub, a makeup remover, a body scrub, a body wash, a hair shampoo, a body shampoo, a leg shaving product, a leather cleaner, or a car upholstery cleaner.

These and other aspects of the present invention will become apparent from the following detailed description of the preferred embodiments.

### DETAILED DESCRIPTION OF THE PREFERRED EMBODIMENTS

A foaming oil of the present invention comprises:

(a) about 60% to about 90%, by weight, of a liquid oil;

(b) about 5% to about 30%, by weight, of a lactylate; and

(c) about 1% to about 8%, by weight, of at least one of an alkanolamide and a sarcosinate,

wherein the composition is essentially free of water.

More particularly, a foaming oil of the present invention comprises about 60% to about 90%, and preferably about 65% to about 85%, by weight of a liquid oil. To achieve the full advantage of the present invention, the composition comprises about 70% to about 85%, by weight, of the liquid oil.

The liquid oil can be a natural oil, a synthetic oil, or a mixture thereof. Examples of natural liquid oils that can be used alone or in admixture, include, but are not limited to, rice bran oil, apricot kernel oil, argan oil, avocado oil, brazil nut oil, canola oil, castor oil, coconut oil, hemp seed oil, lanolin oil, mink oil, macadamia nut oil, olive oil, safflower seed oil, sweet almond oil, sesame seed oil, sunflower seed oil, shea oil, jojoba seed oil, soybean oil, wheat germ oil, and mixtures thereof.

Examples of synthetic oils that can be used alone or in admixture, or in admixture with a natural oil include, but are not limited to, water-insoluble esters. Suitable esters include those comprising an aliphatic alcohol having about eight to about twenty carbon atoms and an aliphatic or aromatic carboxylic acid including from two to about twelve carbon atoms, or conversely, an aliphatic alcohol having two to about twelve carbon atoms with an aliphatic or aromatic carboxylic acid including about eight to about twenty carbon atoms. The ester is either straight-chained or branched. Suitable esters, therefore, include, for example, but are not limited to:

(a) aliphatic monohydric alcohol esters, including, but not limited to:

myristyl propionate, isopropyl isostearate, isopropyl myristate, isopropyl palmitate, ethylhexyl palmitate, cetyl acetate, cetyl propionate, cetyl stearate, isodecyl neopentanoate, cetyl octanoate, isocetyl stearate, octyl stearate;

(b) aliphatic di- and tri-esters of polycarboxylic acid, including, but not limited to:

trimethylpropane, tricaprylate/tricaprate, caprylic/capric triglyceride, octyldodecyl myristate, diisopropyl adipate, diisostearyl fumarate, dioctyl adipate, and triisostearyl citrate;

(c) aliphatic polyhydric alcohol esters, including, but not limited to:

propylene glycol dipelargonate;

(d) aliphatic esters of aromatic acids, including, but not limited to:

C₁₂-C₁₅ alcohol esters of benzoic acid, octyl salicylate, sucrose benzoate, and dioctyl phthalate.

In addition to the liquid oil, a present foaming oil composition comprises about 5% to about 30%, and preferably about 10% to about 30%, by weight, of the composition of a lactylate. To achieve the full advantage of the present invention, the foaming oil composition comprises about 15% to about 30%, by weight, of a lactylate.

Examples of a lactylate that can be used alone or in admixture include, but are not limited to, sodium caproyl lactylate, sodium lauroyl lactylate, sodium stearoyl lactylate, and sodium isostearoyl lactylate.

In addition to the liquid oil and lactylate, a present foaming oil composition comprises about 1% to about 8%, and preferably about 2% to about 7%, by weight of the composition, of at least one of an alkanolamide and/or a sarcosinate. To achieve the full advantage of the present invention, a present foaming oil composition comprises about 1.5% to about 5%, by weight of the composition, of the alkanolamide and/or sarcosinate.

In some embodiments, a present composition contains an alkanolamide and is free of a sarcosinate. In other embodiments, the composition contains a sarcosinate and is free of an alkanolamide. In yet other embodiments, the composition contains both an alkanolamide and a sarcosinate. Persons skilled in the art can select the individual amounts of alkanolamide and/or sarcosinate to achieve a desired composition viscosity, foam volume, and rinsibility.

The alkanolamide is not particularly limited, except that the alkanolamide is a liquid at 25° C. The alkanolamide can be a monoalkanolamide, a dialkanolamide, or a mixture thereof. In some preferred embodiments, the alkanolamide is a monoethanolamide, a diethanolamide, a monoisopropanolamine, or a mixture thereof. The alkanolamide can be one or more of a C₆ to C₂₀ carboxylic acid alkanolamide.

Nonlimiting examples of alkanolamides include, but are not limited to, capramide MEA, capramide DEA, capramide MIPA, cocamide MEA, cocamide DEA, cocamide MIPA, lauramide MIPA, lauramide MEA, lauramide DEA, linoleamide DEA, myristamide DEA, oleamide DEA, palmamide DEA, palmitamide DEA, ricinoleamide DEA, soyamide DEA, undecylenamide DEA, cocoyl sarcosinamide DEA, isostearamide DEA, lanolinamide DEA, lecithinamide DEA, linoleamide DEA, palm kernelamide DEA, tallamide DEA, tallowamide DEA, and mixtures thereof. One preferred alkanolamide is lauramide DEA.

Similarly, the identity of the sarcosinate is not limited, and can be, for example, sodium lauroyl sarcosinate, sodium sarcosinate, sodium cocoyl sarcosinate, sodium myristyl sarcosinate, or any mixture thereof.

In accordance with an important feature of the present invention, a present foaming oil composition is essentially free of water. The term “essentially free” as used herein means that the composition contains less than 0.5%, less than 0.4%, less than 0.3%, less than 0.2%, or less than 0.1%, by weight water. In preferred embodiments, a present composition is free of water, i.e., contains 0% by weight water. Water content is measured using the quantitative Karl Fisher reagent analytical method (ASTM D1744).

In the preparation of a present composition, even small amounts of water are not intentionally added to the composition. Any small amount of water present in the composition typically is present as a result of being a by-product or contaminant in one or more ingredients of the composition.

Because the present compositions are free or essentially free of water, the inclusion of a preservative in the composition is optional. Therefore, one optional ingredient is a preservative, such as phenoxyethanol in an amount of 0% to about 0.25%, by weight, of the composition.

The foaming oil compositions can contain additional optional ingredients. For example, the composition can contain a low amount, e.g., 0% up to about 3%, of an organic solvent. The organic solvent can be used to clarify the composition or adjust composition viscosity. The optional solvent can contain one to six, and typically one to three, hydroxyl groups, e.g., alcohols, diols, triols, and polyols. Specific examples of solvents include, but are not limited to, methanol, ethanol, isopropyl alcohol, n-butanol, n-propyl alcohol, ethylene glycol, propylene glycol, glycerol, diethylene glycol, dipropylene glycol, tripropylene glycol, hexylene glycol, butylene glycol, 1,2,6-hexanetriol, sorbitol, PEG-4, 1,5-pentanediol, similar hydroxyl-containing compounds, and mixtures thereof. The solvent also can be an aprotic solvent, e.g., dimethyl sulfoxide or tetrahydrofuran.

A present foaming oil composition also can contain a gelling agent in an amount of 0% to about 2%, by weight, of the composition. Useful gelling agents are compounds capable of thickening a non-aqueous composition, and include, but are not limited to abietyl alcohol, acrylinoleic acid, aluminum behenate, aluminum caprylate, aluminum dilinoleate, aluminum distearate, aluminum isostearates/laurates/palmitates or stearates, aluminum isostearates/myristates, aluminum isostearates/palmitates, aluminum isostearates/stearates, aluminum lanolate, aluminum myristates/palmitates, aluminum stearate, aluminum stearates, aluminum tristearate, beeswax, behenamide, behenyl alcohol, butadiene/acrylonitrile copolymer, a C₂₉₋₇₀ acid, calcium behenate, calcium stearate, candelilla wax, carnauba, ceresin, cholesterol, cholesteryl hydroxystearate, coconut alcohol, copal, diglyceryl stearate malate, dihydroabietyl alcohol, dimethyl lauramine oleate, dodecanedioic acid/cetearyl alcohol/glycol copolymer, erucamide, ethylcellulose, glyceryl triacetyl hydroxystearate, glyceryl triacetyl ricinoleate, glycol dibehenate, glycol dioctanoate, glycol distearate, hexanediol distearate, hydrogenated C₆₋₁₄ olefin polymers, hydrogenated castor oil, hydrogenated cottonseed oil, hydrogenated lard, hydrogenated menhaden oil, hydrogenated palm kernel glycerides, hydrogenated palm kernel oil, hydrogenated palm oil, hydrogenated polyisobutene, hydrogenated soybean oil, hydrogenated tallow amide, hydrogenated tallow glyceride, hydrogenated vegetable glyceride, hydrogenated vegetable glycerides, hydrogenated vegetable oil, hydroxypropylcellulose, isobutylene/isoprene copolymer, isocetyl stearoyl stearate, Japan wax, jojoba wax, lanolin alcohol, lauramide, methyl dehydroabietate, methyl hydrogenated rosinate, methyl rosinate, methylstyrene/vinyltoluene copolymer, microcrystalline wax, montan acid wax, montan wax, myristyleicosanol, myristyloctadecanol, octadecene/maleic anhydride copolymer, octyldodecyl stearoyl stearate, oleamide, oleostearine, ouricury wax, oxidized polyethylene, ozokerite, palm kernel alcohol, paraffin, pentaerythrityl hydrogenated rosinate, pentaerythrityl rosinate, pentaerythrityl tetraabietate, pentaerythrityl tetrabehenate, pentaerythrityl tetraoctanoate, pentaerythrityl tetraoleate, pentaerythrityl tetrastearate, phthalic anhydride/glycerin/glycidyl decanoate copolymer, phthalic/trimellitic/glycols copolymer, polybutene, polybutylene terephthalate, polydipentene, polyethylene, polyisobutene, polyisoprene, polyvinyl butyral, polyvinyl laurate, propylene glycol dicaprylate, propylene glycol dicocoate, propylene glycol diisononanoate, propylene glycol dilaurate, propylene glycol dipelargonate, propylene glycol distearate, propylene glycol diundecanoate, PVP/eicosene copolymer, PVP/hexadecene copolymer, rice bran wax, stearalkonium bentonite, stearalkonium hectorite, stearamide, stearamide DEA-distearate, stearamide DIBA-stearate, stearamide MEA-stearate, stearone, stearyl alcohol, stearyl erucamide, stearyl stearate, stearyl stearoyl stearate, synthetic beeswax, synthetic wax, trihydroxystearin, triisononanoin, triisostearin, triisostearyl trilinoleate, trilaurin, trilinoleic acid, trilinolein, trimyristin, triolein, tripalmitin, tristearin, zinc laurate, zinc myristate, zinc neodecanoate, zinc rosinate, zinc stearate, and mixtures thereof.

Other classes of optional ingredients included in a present foaming oil composition can be, but not limited to, dyes, fragrances, opacifiers, and similar classes of ingredients known to persons skilled in the art. These optional ingredients are included in the composition in a sufficient amount to perform their intended function without adversely affecting the beneficial properties of the composition.

The present compositions exhibit the properties of a non-aqueous composition, while possessing the rinsibility properties of an aqueous composition, thereby allowing the composition to be rinsed from a surface with water. The important benefits of the present compositions is the ability to remove oily residues, like facial makeup and oily debris, wherein the composition cuts through the residues, and also possess the water rinsibility to be easily removed by a water wash.

The present compounds therefore exhibit the beneficial properties of both an oil-based composition and an aqueous composition, while overcoming the disadvantages of these classes of compounds, i.e., the harshness of aqueous compositions and the lack of rinsibility of oil-based compositions.

The present compositions therefore can be formulated as a hair shampoo, a body shampoo, foaming bath oil, makeup remover, facial cleanser, ladies' leg shave, men's facial shave, facial scrub, body scrub, leather cleaner, and wood cleaner/polish.

A composition of the present invention is prepared by simply admixing the composition ingredients. In some embodiments, preblends are utilized in the preparation of the compositions.

In various embodiments, a present composition is prepared by blending the ingredients in any order.

### EXAMPLE 1

| Ingredients | % w/w |
|---|---|
| Sweet Almond Oil | 71 |
| Sodium Caproyl Lactylate | 25 |
| Lauramide DEA | 3 |
| Fragrance | 1 |

### EXAMPLE 2

| Ingredients | % w/w |
|---|---|
| Sweet Almond Oil | 76 |
| Sodium Caproyl Lactylate | 7 |
| Sodium Lauroyl Lactylate | 13 |
| Sodium Lauroyl Sarcosinate | 3 |
| Fragrance | 1 |

### EXAMPLE 3

| Ingredients | % w/w |
|---|---|
| Sweet Almond Oil | 83 |
| Sodium Caproyl Lactylate | 4 |
| Sodium Lauroyl Lactylate | 5 |
| Sodium Lauroyl Sarcosinate | 2 |
| Cocamide DEA | 5 |
| Fragrance | 1 |

A foaming oil composition of the present invention is applied to an animate or inanimate surface, typically by rubbing, spraying, or use of an applicator. It is not necessary to dilute the composition. Typically, a present foaming oil composition is used “as is” for application to the surface.

A simple application of the composition provides the cleaning action. A preferred route of administration is rubbing the composition onto the surface with a soft massage to ensure intimate contact with the surface, such as, the skin. In some embodiments, no mechanical scrubbing or rubbing is necessary to remove dirt, grease, and oil from the treated surface. The composition then is simply water rinsed from the surface. The resulting surface is clean, and free of oily residues.

A present composition exhibits unique properties including a continuous oil phase that is compatible with the lipids of the skin, that does not strip the lipids, and that also can emulsify and foam. A present composition can act as a water-resistant to waterproof makeup remover, acting as an emulsifier with very mild and low foaming properties, or as a hair and body shampoo with a copious foam and excellent hair and skin conditioning feel due to the particular oil used in the formulation. Such applications are unique in that no water is utilized, but solely oils and anhydrous emulsifiers and surfactants, i.e., the lactylates, which act as a coupling agent to compatibilize the oils and anhydrous surfactants, and permit the oil to be spread, then gently foamed away with a water rinse.

Obviously, many modifications and variations of the invention as hereinbefore set forth can be made without departing from the spirit and scope thereof, and, therefore, only such limitations should be imposed as are indicated by the appended claims

What is claimed:

1. A composition comprising
(a) about 60% to about 90%, by weight, of a liquid oil;
(b) about 5% to about 30%, by weight, of a lactylate; and
(c) about 1% to about 8%, by weight, of at least one of an alkanolamide and a sarcosinate,
wherein the composition is essentially free of water.

2. The composition of claim 1 wherein the liquid oil is present in an amount of about 65% to about 85%, by weight, of the composition.

3. The composition of claim 1 wherein the liquid oil is a natural oil, a synthetic oil, or a mixture thereof.

4. The composition of claim 1 wherein the lactylate is present in an amount of about 10% to about 30%, by weight, for the composition.

5. The composition of claim 1 wherein the lactylate comprises sodium caproyl lactylate, sodium lauroyl lactylate, sodium stearoyl lactylate, and sodium isostearoyl lactylate, or mixtures thereof.

6. The composition of claim 1 wherein the at least one alkanolamide and sarconsinate is present in an amount of about 2% to about 7%, by weight, of the composition.

7. The composition according to claim 1 wherein the composition comprises an alkanolamide.

8. The composition according to claim 1 wherein the composition comprises a sarcosinate.

9. The composition according to claim 1 wherein the composition comprises an alkanolamide and a sarcosinate.

10. The composition according to claim 1 wherein the alkanolamide comprises a monoalkanolamide, a dialkanolamide, or a mixture thereof.

11. The composition according to claim 1 wherein the alkanolamide is one or more C₆ to C₂₀ carboxylic acid alkanolamide.

12. The composition according to claim 1 wherein the alkanolamide is a monoethanolamide, a diethanolamide, a monoisopropanolamine, or a mixture thereof.

13. The composition of claim 1 wherein the alkanolamine comprises capramide MEA, capramide DEA, capramide MIPA, cocamide MEA, cocamide DEA, cocamide MIPA, lauramide MIPA, lauramide MEA, lauramide DEA, linoleamide DEA, myristamide DEA, oleamide DEA, palmamide DEA, palmitamide DEA, ricinoleamide DEA, soyamide DEA, undecylenamide DEA, cocoyl sarcosinamide DEA, isostearamide DEA, lanolinamide DEA, lecithinamide DEA, linoleamide DEA, palm kernelamide DEA, tallamide DEA, tallowamide DEA, and mixtures thereof.

14. The composition of claim 1 wherein the sarcosinate comprises sodium lauroyl sarcosinate, sodium sarcosinate, sodium cocoyl sarcosinate, sodium myristyl sarcosinate, or a mixture thereof.

15. The composition of claim 1 wherein the composition comprises less than 0.4%, by weight, of water.

16. The composition of claim 1 wherein the composition comprises less than 0.2%, by weight, of water.

17. The composition of claim 1 wherein the composition is free of water.

18. A method of cleaning an animate or inanimate surface comprising applying a composition of claim 1 to the surface, then rinsing the composition from the surface with water to generate a foam.

19. The method of claim 18 wherein the inanimate surface is a metal, a plastic, a mineral surface, a glass, wood, or leather.

20. The method of claim 18 wherein the animate surface is skin or hair.

21. A method of removing makeup from skin comprising applying a composition of claim 1 to the skin, then contacting the skin with a sufficient amount of water to generate a foam and dislodge the makeup from the skin.
